(19) United States
(12) Patent Application Publication
(10) Pub. No.: US 2023/0341292 A1
(43) Pub. Date: Oct. 26, 2023

(54) ON-WAFER TEST MECHANISM FOR WAVEGUIDES

(71) Applicant: GOOGLE LLC, Mountain View, CA (US)

(72) Inventor: Joseph Daniel Lowney, Tucson, AZ (US)

(21) Appl. No.: 18/136,798

(22) Filed: Apr. 19, 2023

Related U.S. Application Data
(60) Provisional application No. 63/332,825, filed on Apr. 20, 2022.

(51) Int. Cl. G01M 11/00

(52) U.S. Cl. CPC G01M 11/338

(57) ABSTRACT

An on-wafer testing mechanism includes multiple waveguides and test structures disposed on a wafer. Light sources are coupled to the wafer and provide beams of light to the structures disposed on the wafer by propagating the light through the wafer. In response to receiving at least a portion of a beam of light, a test structure is configured to guide the light to an exit location on the test structure. As light exits a test structure, a conoscope determines the diffraction efficiency of the test structure based on a measurement taken of the light exiting the test structure.

### CROSS-REFERENCE TO RELATED APPLICATIONS

The present application claims priority to U.S. Provisional Patent Application Ser. No. 63/332,825, entitled “ON-WAFER TEST MECHANISM FOR WAVEGUIDES” and filed on Apr. 20, 2022, the entirety of which is incorporated by reference herein.

### BACKGROUND

Some head-worn displays (HWDs) include waveguides configured to direct light from an optical engine to the eye of the user in order to display one or more images to the user. For example, some waveguides include grating structures to expand the exit pupil of light received from an optical engine and then provide the light with the expanded exit pupil to the eye of a user. These waveguides are commonly fabricated on wafers (e.g., 150 mm, 200 mm, or 300 mm wafers) with such wafers often including 10 or more waveguides to increase the efficiency of the fabrication process. To test the fidelity of the waveguides after fabrication, testing procedures include first dicing the wafers and then performing metrology processes on the diced portions of the wafer. Such metrology processes include, for example, scanning electro-microscopy, atomic force microscopy, or an optical characterization conducted by coupling a waveguide to an optical projection system. However, such testing procedures are complex and time-consuming, leading to increased costs in the fabrication of the waveguides.

### BRIEF DESCRIPTION OF THE DRAWINGS

The present disclosure may be better understood, and its numerous features and advantages are made apparent to those skilled in the art by referencing the accompanying drawings. The use of the same reference symbols in different drawings indicates similar or identical items.

FIG. 1 is a diagram of an example display system housing a laser projector system configured to project images toward the eye of a user, in accordance with some embodiments.

FIG. 2 is a diagram illustrating a laser projection system that projects images directly onto the eye of a user via laser light, in accordance with some embodiments.

FIG. 3 is a diagram illustrating an example waveguide exit pupil expansion system, in accordance with embodiments.

FIG. 4 is a block diagram of an on-wafer test mechanism for testing the fidelity of structures on a wafer, in accordance with some embodiments.

FIG. 5 is a block diagram of an on-wafer test mechanism using light provided by a light-emitting diode to test the fidelity of structures of a wafer, in accordance with some embodiments.

FIG. 6 is a k-space mode diagram demonstrating an example collimation of light within an on-wafer test mechanism, in accordance with some embodiments.

FIG. 7 is a block diagram of a cross-sectional view of an on-wafer test mechanism for waveguides including a conoscope, in accordance with some embodiments.

FIG. 8 is a block diagram of a cross-sectional view of an on-wafer test mechanism including a conoscope configured to detect multiple wavelengths of light, in accordance with some embodiments.

FIG. 9 is a flow diagram of an example method for testing the fidelity of structures on a wafer, in accordance with some embodiments.

### DETAILED DESCRIPTION

Some head-worn devices (HWDs) (e.g., augmented reality head-worn devices) are designed to look like eyeglasses, with at least one of the lenses containing a waveguide to direct light to a user's eye. The combination of the lens and waveguide is referred to as an “optical combiner”. Such waveguides form, for example, exit pupil expanders (EPEs) and outcouplers that form and guide light from an optical engine (e.g., projector) to the user's eye. The HWDs generally have a frame designed to be worn in front of a user's eyes to allow the user to view both their environment and computer-generated content projected from the combiner. Components that are necessary to the functioning of a typical HWDs, such as, for example, an optical engine to project computer-generated content (e.g., light representative of one or more images), cameras to pinpoint physical location, cameras to track the movement of the user's eye(s), processors to power the optical engine, and a power supply, are typically housed within the frame of the HWD. As an HWD frame has limited volume in which to accommodate these components, it is desirable that these components be as small as possible and configured to interact with the other components in very small volumes of space.

To fabricate one or more waveguides for an HWD, one or more wafers (e.g., glass wafers, plastic wafers) each forming (e.g., including) one more waveguides are produced. For example, to increase the efficiency of the fabrication process, a wafer including 10 or more waveguides is produced. Each wafer, for example, includes two opposing surfaces and has a shape having at least one flat edge. The waveguides of a wafer each include one or more respective sets of gratings (e.g., Bragg grating structures, surface-relief grating structures, polarization volume grating structures, volumetric holographic grating structures) disposed on the surface of the waveguide (e.g., disposed on the surface of the wafer) and forming an incoupler, EPE, outcoupler, or any combination thereof of the waveguide. To help test the fidelity of these gratings of the waveguides, the wafer including the waveguides has one or more test structures fabricated on a surface of the wafer along with the gratings of the waveguides. That is to say, a wafer includes one or more test structures disposed on the surface of the wafer and one or more sets of gratings for one or more waveguides disposed on the surface of the wafer. These test structures include, for example, sets of test gratings (e.g., linear gratings) configured to guide light from a first point (e.g., entry point) of the test structure to a second location (e.g., exit location) on the test structure.

To test the integrity of the gratings of the waveguides disposed on the wafer (e.g., test the structures on the wafer), one or more light sources (e.g., light-emitting diodes, laser diodes) are disposed proximate to or otherwise coupled to the wafer. For example, the light sources are disposed proximate to or otherwise coupled to a flat edge of the wafer. Such light sources, for example, are configured to emit light (e.g., beams of light) each associated with one or more wavelengths. As an example, such light sources are configured to emit white light, red light, green light, blue light, or any combination thereof. Because the light sources are disposed proximate to or otherwise coupled to the flat edge of the wafer, the light sources emit light such that the light propagates through the waveguide using total internal reflection (TIR), partial internal reflection (PIR), or both. As the light propagates through the wafer, the light is received by a set of test gratings of a test structure. That is to say, the light is received at a first point (e.g., entry point) of the test structure. In response to receiving the light, the test gratings of a test structure guide the light from the first point of the test structure to a second point (e.g., exit point) of the test structure and out of the wafer. As light exits the wafer at the exit point of the test structure, a conoscope takes one or more measurements of the light and determines the grating efficiency of the test gratings of the structure based on one or more of the measurements. In this way, the fidelity of the structures (e.g., test gratings, gratings of the waveguides) on the surface of a wafer are tested without first dicing the wafer or performing complex microscopy. As such, the time and cost needed to test the gratings of the wafer are reduced.

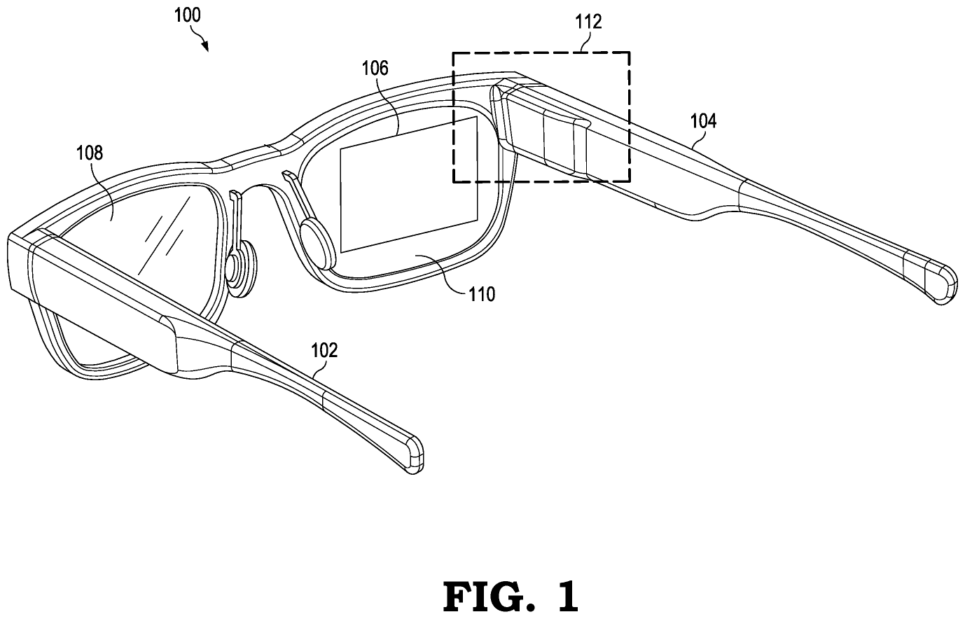

FIG. 1 illustrates an example display system 100 having a support structure 102 that includes an arm 104, which houses a laser projection system configured to project images toward the eye of a user, such that the user perceives the projected images as being displayed in a field of view (FOV) area 106 of a display at one or both of lens elements 108, 110. In the depicted embodiment, the display system 100 is an eyewear display that includes a support structure 102 configured to be worn on the head of a user and has a general shape and appearance of an eyeglasses (e.g., sunglasses) frame. The support structure 102 contains or otherwise includes various components to facilitate the projection of such images toward the eye of the user, such as a laser projector, an optical scanner, and a waveguide. In some embodiments, the support structure 102 further includes various sensors, such as one or more front-facing cameras, rear-facing cameras, other light sensors, motion sensors, accelerometers, and the like. The support structure 102 further can include one or more radio frequency (RF) interfaces or other wireless interfaces, such as a Bluetooth™ interface, a Wi-Fi interface, and the like. Further, in some embodiments, the support structure 102 further includes one or more batteries or other portable power sources for supplying power to the electrical components of the display system 100. In some embodiments, some or all of these components of the display system 100 are fully or partially contained within an inner volume of support structure 102, such as within the arm 104 in region 112 of the support structure 102. It should be noted that while an example form factor is depicted, it will be appreciated that in other embodiments the display system 100 may have a different shape and appearance from the eyeglasses frame depicted in FIG. 1.

One or both of the lens elements 108, 110 are used by the display system 100 to provide an augmented reality (AR) display in which rendered graphical content can be superimposed over or otherwise provided in conjunction with a real-world view as perceived by the user through the lens elements 108, 110. For example, laser light used to form a perceptible image or series of images may be projected by a laser projector of the display system 100 onto the eye of the user via a series of optical elements, such as a waveguide formed at least partially in the corresponding lens element, one or more scan mirrors, and one or more optical relays. One or both of the lens elements 108, 110 thus include at least a portion of a waveguide that routes display light received by an incoupler of the waveguide to an outcoupler of the waveguide, which outputs the display light toward an eye of a user of the display system 100. The display light is modulated and scanned onto the eye of the user such that the user perceives the display light as an image. In addition, each of the lens elements 108, 110 is sufficiently transparent to allow a user to see through the lens elements to provide a field of view of the user's real-world environment such that the image appears superimposed over at least a portion of the real-world environment.

In some embodiments, the projector is a digital light processing-based projector, a scanning laser projector, or any combination of a modulative light source such as a laser or one or more LEDs and a dynamic reflector mechanism such as one or more dynamic scanners or digital light processors. In some embodiments, the projector includes multiple laser diodes (e.g., a red laser diode, a green laser diode, and/or a blue laser diode) and at least one scan mirror (e.g., two one-dimensional scan mirrors, which may be micro-electromechanical system (MEMS)-based or piezo-based). The projector is communicatively coupled to the controller and a non-transitory processor-readable storage medium or memory storing processor-executable instructions and other data that, when executed by the controller, cause the controller to control the operation of the projector. In some embodiments, the controller controls a scan area size and scan area location for the projector and is communicatively coupled to a processor (not shown) that generates content to be displayed at the display system 100. The projector scans light over a variable area, designated the FOV area 106, of the display system 100. The scan area size corresponds to the size of the FOV area 106 and the scan area location corresponds to a region of one of the lens elements 108, 110 at which the FOV area 106 is visible to the user. Generally, it is desirable for a display to have a wide FOV to accommodate the outcoupling of light across a wide range of angles. Herein, the range of different user eye positions that will be able to see the display is referred to as the eyebox of the display.

In some embodiments, the projector routes light via first and second scan mirrors, an optical relay disposed between the first and second scan mirrors, and a waveguide disposed at the output of the second scan mirror. In some embodiments, at least a portion of an outcoupler of the waveguide may overlap the FOV area 106. These aspects are described in greater detail below.

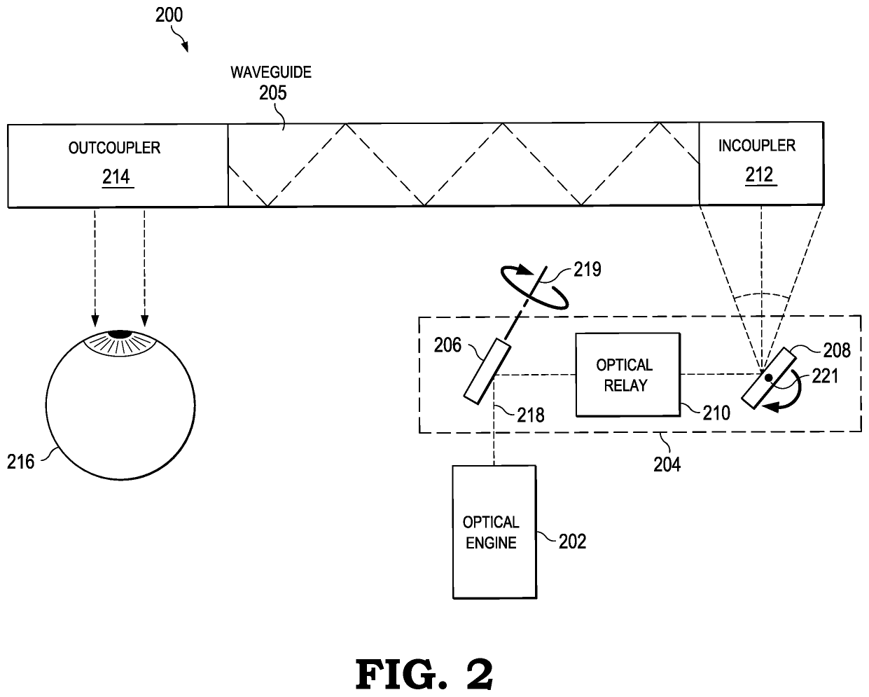

FIG. 2 illustrates a simplified block diagram of a laser projection system 200 that projects images directly onto the eye of a user via laser light. The laser projection system 200 includes an optical engine 202, an optical scanner 204, and a waveguide 205. The optical scanner 204 includes a first scan mirror 206, a second scan mirror 208, and an optical relay 210. The waveguide 205 includes an incoupler 212 and an outcoupler 214, with the outcoupler 214 being optically aligned with an eye 216 of a user in the present example. In some embodiments, the laser projection system 200 is implemented in a wearable heads-up display or other display system, such as the display system 100 of FIG. 1.

The optical engine 202 includes one or more laser light sources configured to generate and output laser light 218 (e.g., visible laser light such as red, blue, and green laser light and/or non-visible laser light such as infrared laser light). In some embodiments, the optical engine 202 is coupled to a driver or other controller (not shown), which controls the timing of emission of laser light from the laser light sources of the optical engine 202 in accordance with instructions received by the controller or driver from a computer processor coupled thereto to modulate the laser light 218 to be perceived as images when output to the retina of an eye 216 of a user.

For example, during the operation of the laser projection system 200, multiple laser light beams having respectively different wavelengths are output by the laser light sources of the optical engine 202, then combined via a beam combiner (not shown), before being directed to the eye 216 of the user. The optical engine 202 modulates the respective intensities of the laser light beams so that the combined laser light reflects a series of pixels of an image, with the particular intensity of each laser light beam at any given point in time contributing to the amount of corresponding color content and brightness in the pixel being represented by the combined laser light at that time.

One or both of the scan mirrors 206 and 208 of the optical scanner 204 are MEMS mirrors in some embodiments. For example, the scan mirror 206 and the scan mirror 208 are MEMS mirrors that are driven by respective actuation voltages to oscillate during active operation of the laser projection system 200, causing the scan mirrors 206 and 208 to scan the laser light 218. Oscillation of the scan mirror 206 causes laser light 218 output by the optical engine 202 to be scanned through the optical relay 210 and across a surface of the second scan mirror 208. The second scan mirror 208 scans the laser light 218 received from the scan mirror 206 toward an incoupler 212 of the waveguide 205. In some embodiments, the scan mirror 206 oscillates along a first scanning axis 219, such that the laser light 218 is scanned in only one dimension (i.e., in a line) across the surface of the second scan mirror 208. In some embodiments, the scan mirror 208 oscillates or otherwise rotates along a second scanning axis 221. In some embodiments, the first scanning axis 219 is perpendicular to the second scanning axis 221.

In some embodiments, the incoupler 212 has a substantially rectangular profile and is configured to receive the laser light 218 and direct the laser light 218 into the waveguide 205. The incoupler 212 is defined by a smaller dimension (i.e., width) and a larger orthogonal dimension (i.e., length). In an embodiment, the optical relay 210 is a line-scan optical relay that receives the laser light 218 scanned in a first dimension by the first scan mirror 206 (e.g., the first dimension corresponding to the small dimension of the incoupler 212), routes the laser light 218 to the second scan mirror 208, and introduces a convergence to the laser light 218 in the first dimension to an exit pupil beyond the second scan mirror 208. Herein, an “exit pupil” in an optical system refers to the location along the optical path where beams of light intersect. For example, the possible optical paths of the laser light 218, following reflection by the first scan mirror 206, are initially spread along the first scanning axis, but later these paths intersect at an exit pupil beyond the second scan mirror 208 due to convergence introduced by the optical relay 210. For example, the width (i.e., smallest dimension) of a given exit pupil approximately corresponds to the diameter of the laser light corresponding to that exit pupil. Accordingly, the exit pupil can be considered a “virtual aperture”. According to various embodiments, the optical relay 210 includes one or more collimation lenses that shape and focus the laser light 218 on the second scan mirror 208 or includes a molded reflective relay that includes two or more spherical, aspheric, parabolic, and/or freeform lenses that shape and direct the laser light 218 onto the second scan mirror 208. The second scan mirror 208 receives the laser light 218 and scans the laser light 218 in a second dimension, the second dimension corresponding to the long dimension of the incoupler 212 of the waveguide 205. In some embodiments, the second scan mirror 208 causes the exit pupil of the laser light 218 to be swept along a line along the second dimension. In some embodiments, the incoupler 212 is positioned at or near the swept line downstream from the second scan mirror 208 such that the second scan mirror 208 scans the laser light 218 as a line or row over the incoupler 212.

In some embodiments, the optical engine 202 includes an edge-emitting laser (EEL) that emits a laser light 218 having a substantially elliptical, non-circular cross-section, and the optical relay 210 magnifies or minimizes the laser light 218 along its semi-major or semi-minor axis to circularize the laser light 218 prior to convergence of the laser light 218 on the second scan mirror 208. In some such embodiments, a surface of a mirror plate of the scan mirror 206 is elliptical and non-circular (e.g., similar in shape and size to the cross-sectional area of the laser light 218). In other such embodiments, the surface of the mirror plate of the scan mirror 206 is circular.

The waveguide 205 of the laser projection system 200 includes the incoupler 212 and the outcoupler 214. The term “waveguide,” as used herein, will be understood to mean a combiner using one or more of total internal reflection (TIR), specialized filters, and/or reflective surfaces, to transfer light from an incoupler (such as the incoupler 212) to an outcoupler (such as the outcoupler 214). In some display applications, the light is a collimated image, and the waveguide transfers and replicates the collimated image to the eye. In general, the terms “incoupler” and “outcoupler” will be understood to refer to any type of optical grating structure, including, but not limited to, diffraction gratings, holograms, holographic optical elements (e.g., optical elements using one or more holograms), volume diffraction gratings, volume holograms, surface relief diffraction gratings, and/or surface relief holograms. In some embodiments, a given incoupler or outcoupler is configured as a transmissive grating (e.g., a transmissive diffraction grating or a transmissive holographic grating) that causes the incoupler or outcoupler to transmit light and to apply designed optical function(s) to the light during the transmission. In some embodiments, a given incoupler or outcoupler is a reflective grating (e.g., a reflective diffraction grating or a reflective holographic grating) that causes the incoupler or outcoupler to reflect light and to apply designed optical function(s) to the light during the reflection. In the present example, the laser light 218 received at the incoupler 212 is relayed to the outcoupler 214 via the waveguide 205 using TIR. The laser light 218 is then output to the eye 216 of a user via the outcoupler 214. As described above, in some embodiments the waveguide 205 is implemented as part of an eyeglass lens, such as the lens 108 or lens 110 (FIG. 1) of the display system having an eyeglass form factor and employing the laser projection system 200.

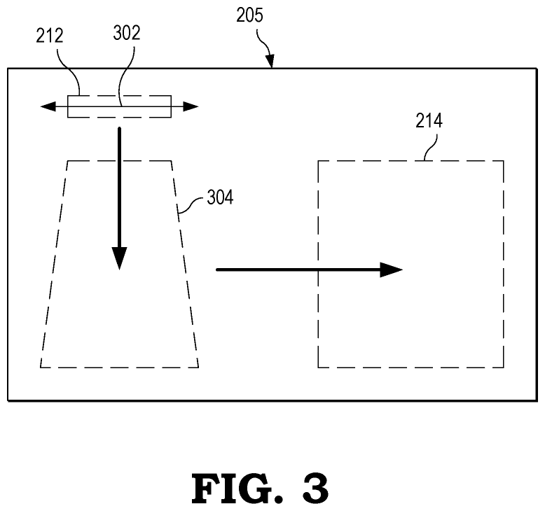

Although not shown in the example of FIG. 2, in some embodiments additional optical components are included in any of the optical paths between the optical engine 202 and the scan mirror 206, between the scan mirror 206 and the optical relay 210, between the optical relay 210 and the scan mirror 208, between the scan mirror 208 and the incoupler 212, between the incoupler 212 and the outcoupler 214, and/or between the outcoupler 214 and the eye 216 (e.g., in order to shape the laser light for viewing by the eye 216 of the user). In some embodiments, a prism is used to steer light from the scan mirror 208 into the incoupler 212 so that light is coupled into incoupler 212 at the appropriate angle to encourage propagation of the light in waveguide 205 by TIR. Also, in some embodiments, an exit pupil expander (e.g., an exit pupil expander 304 of FIG. 3, described below), such as a fold grating, is arranged in an intermediate stage between incoupler 212 and outcoupler 214 to receive light that is coupled into waveguide 205 by the incoupler 212, expand the light, and redirect the light towards the outcoupler 214, where the outcoupler 214 then couples the laser light out of waveguide 205 (e.g., toward the eye 216 of the user).

FIG. 3 shows an example of light propagation within the waveguide 205 of the laser projection system 200 of FIG. 2 in accordance with some embodiments. As shown, light received via the incoupler 212, which is scanned along the scanning axis 302, is directed into an exit pupil expander (EPE) 304 and is then routed to the outcoupler 214 to be output (e.g., toward the eye of the user). In some embodiments, the exit pupil expander 304 expands one or more dimensions of the eyebox of a WHUD that includes the laser projection system 200 (e.g., with respect to what the dimensions of the eyebox of the WHUD would be without the exit pupil expander 304). In some embodiments, the incoupler 212 and the exit pupil expander 304 each include respective one-dimensional diffraction gratings (i.e., diffraction gratings that extend along one dimension). It should be understood that FIG. 3 shows a substantially ideal case in which the incoupler 212 directs light straight down (with respect to the presently illustrated view) in a first direction that is perpendicular to the scanning axis 302, and the exit pupil expander 304 directs light to the right (with respect to the presently illustrated view) in a second direction that is perpendicular to the first direction. While not shown in the present example, it should be understood that, in some embodiments, the first direction in which the incoupler 212 directs light is slightly or substantially diagonal, rather than exactly perpendicular, with respect to the scanning axis 302.

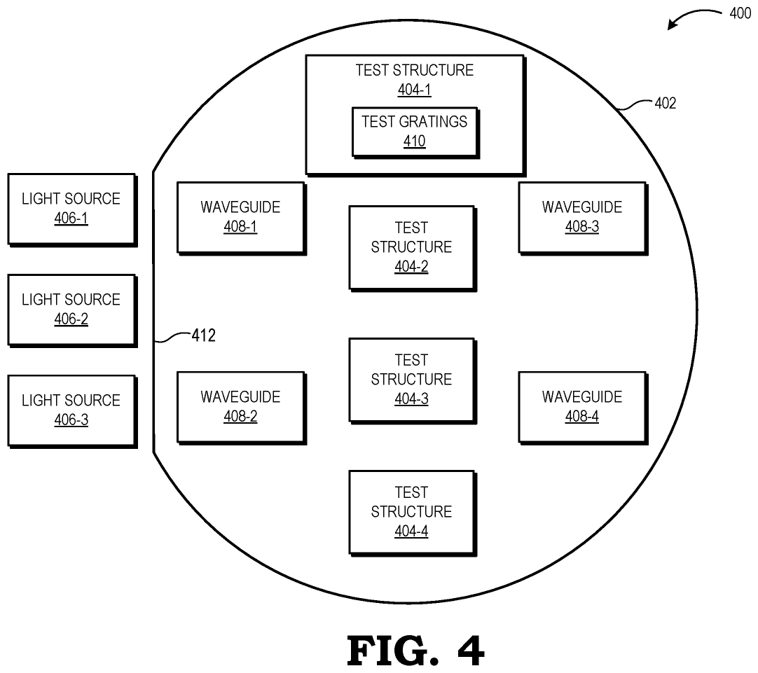

In embodiments, waveguides such as waveguide 205 are fabricated on one or more wafers (e.g., glass wafers, plastic wafers) with each wafer forming at least a portion of one or more waveguides. Further, for each waveguide at least partially formed by a wafer, the wafer includes one or more respective sets of gratings disposed on the surface of the wafer that form the incoupler, EPE, outcoupler, or any combination of a respective waveguide. To test the fidelity of the gratings on the wafer, some testing procedures include first dicing the wafers into two or more portions and then performing metrology processes (e.g., scanning electro-microscopy, atomic force microscopy, optical characterization) on each of the diced portions of the wafer. However, first dicing the wafer before testing the gratings of the waveguides is time-consuming, leading to increased costs in the fabrication of the waveguides. To this end, FIG. 4 presents an on-wafer test mechanism 400 for a wafer including one or more waveguides. In embodiments, wafer 402 (e.g., a glass wafer, a plastic wafer) of on-wafer test mechanism 400 includes opposing surfaces and has a shape having at least one flat edge 412. Further, wafer 402 forms at least a portion of one or more waveguides 408, each similar to or the same as waveguide 205. As an example, wafer 402 forms at least a portion of waveguides 408-1, 408-2, 408-3, and 408-4. Though the example embodiment in FIG. 4 presets wafer 402 as including (e.g., forming at least a portion of) four waveguides (408-1, 408-2, 408-3, 408-4), in other embodiments, wafer 402 may include any number of waveguides 408. In embodiments, wafer 402 is configured to propagate light through the interior of wafer 402 using TIR, PIR, or both, similar to or the same as waveguide 205.

According to embodiments, each waveguide 408 of the wafer includes one or more respective sets of gratings (e.g., Bragg gratings, surface-relief gratings, polarization volume gratings, volumetric holographic gratings) disposed on the surface of the waveguide 408 (e.g., disposed on the surface of the wafer 402 forming at least a portion of the waveguide 408). The sets of gratings for each waveguide 408 respectively form the incoupler (e.g., incoupler 212), EPE (e.g., exit pupil expander 304), outcoupler (e.g., outcoupler 214), or any combination thereof of the waveguide 408. To help test the fidelity of these gratings of the waveguides 408, the wafer 402 of on-wafer test mechanism 400 includes one or more test structures 404 disposed on a surface of the wafer 402. According to some embodiments, test structures 404 are fabricated on wafer 402 concurrently with one or more waveguides 408 of wafer 402. Though the example embodiment in FIG. 4 presents wafer 402 as including four test structures (404-1, 404-2, 404-3, 404-4), in other embodiments, wafer 402 can include any number of test structures 404. In embodiments, each test structure 404, includes, for example, one or more sets of test gratings 410 (e.g., linear gratings, Bragg gratings, surface-relief gratings, polarization volume gratings, volumetric holographic gratings) configured to guide light propagating within wafer 402 from a first point (e.g., entry point) of a test structure 404 to a second point (e.g., exit point) on the test structure 404 such that the light at the exit point exits wafer 402. That is to say, the test gratings 410 of a test structure 404 are configured to guide light (e.g., white light, red light, blue light, green light) propagating within wafer 402 out of wafer 402.

Further, to test the fidelity of the gratings of one or more waveguides 408 of wafer 402, on-wafer test mechanism 400 includes one or more light sources 406 each configured to provide light (e.g., white light, blue light, red light, green light) to one or more test structures 404. For example, one or more light sources 406 are configured to provide light to a test structure 404 by providing light to wafer 402 such that the light propagates through wafer 402 until it is received at the test structure 404. Such light sources 406, for example, include one or more light-emitting diodes (LEDs), laser diodes, or both. In embodiments, one or more light sources 406 are disposed proximate to or otherwise coupled to the flat edge 412 of wafer 402 such that light emitted from the light sources 406 propagates through wafer 402. As an example, a light source 406 is disposed proximate to the flat edge 412 of wafer 402 such that the light source 406 is at a distance from the flat edge 412 that allows the light source 406 to provide light to one or more test structures 404 disposed on wafer 402 by propagating light in wafer 402. Though the example embodiment illustrated in FIG. 4 presents three light sources (406-1, 406-2, 406-3) disposed proximate to or otherwise coupled to the flat edge 412 of wafer 402, in other embodiments any number of light sources 406 may be disposed proximate to or otherwise coupled to the flat edge 412 of wafer 402.

According to embodiments, in response to receiving light from one or more light sources 406, the test gratings 410 of a test structure 404 direct the light from a first point (e.g., entry point) on the test structure 404 to a second point (e.g., exit point) on the test structure 404 such that the light exits wafer 402. That is to say, in response to receiving light from one or more light sources 406, the test gratings of a test structure 404 direct the light out of wafer 402. In embodiments, the angles (e.g., angular distribution), intensity, or both of the light directed out of the wafer 402 by a set of test gratings 410 is measured, for example, by a conoscope. Based on the measured angles, angular distribution, intensity, or any combination thereof, the conoscope determines a diffraction efficiency for the test gratings 410 that directed the light out of the wafer 402. Such a determined diffraction efficiency, indicates, for example, the fidelities of all the gratings (e.g., test gratings 410 and gratings of the waveguides 408) on the wafer 402. For example, the determined diffraction efficiency being below a predetermined threshold value indicates a low fidelity for the gratings (e.g., test gratings 410 and gratings of the waveguides 408) on the wafer 402. As another example, the determined diffraction efficiency being equal to or higher than a predetermined threshold value indicates a high fidelity for the gratings (e.g., test gratings 410 and gratings of the waveguides 408) on the wafer 402. In this way, the fidelities of the gratings of the waveguides 408 are determined without first dicing up wafer 402, simplifying the process to test the gratings and lowering the time and cost needed to test the gratings.

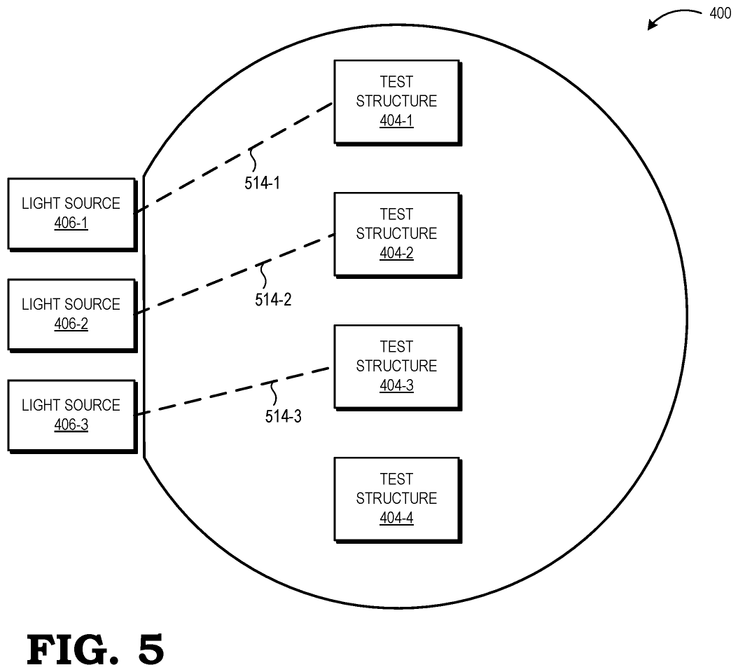

Referring now to FIG. 5, a block diagram of an on-wafer test mechanism 400 using light provided by a light source to test the fidelity of the gratings (e.g., structures) on a wafer is presented. According to embodiments, to test the fidelity of the gratings fabricated on wafer 402 (e.g., the gratings of waveguides 408), one or more light sources 406 are activated concurrently, sequentially (e.g., in a sweep), or both. After one or more light sources 406 are activated, the activated light sources 406 then provide one or more beams of light 514-1, 514-2, 514-3 (e.g., white light, red light, blue light, green light) to one or more test structures 404. For example, the activated light sources 406 provide one or more beams of light 514 to wafer 402 such that at least a portion of the beams of light 514 propagate through wafer 402 (e.g., by TIR, PIR, or both) and are received at one or more test structures 404. In response to receiving at least a portion of a beam of light 514 provided from one or more light sources 406, a test structure 404 is configured to direct at least a portion of the received light out of wafer 402 (e.g., in a direction perpendicular to the surface of wafer 402). As an example, in response to receiving at least a portion of a beam of light 514 provided from one or more light sources 406, a set of test gratings 410 of a test structure 404 is configured to direct at least a portion of the received light out of wafer 402. After the received light is directed out of wafer 402, the intensity of the light exiting wafer 402 is measured to determine the fidelity of the gratings (e.g., test gratings 410, gratings of the waveguides 408) on wafer 402. Though the example embodiment presented in FIG. 5 illustrates each light source (406-1, 406-2, 406-3) each emitting one beam of light (514-1, 514-2, 514-3), in other embodiments, any number of light sources 406 may emit any number of beams of light 514.

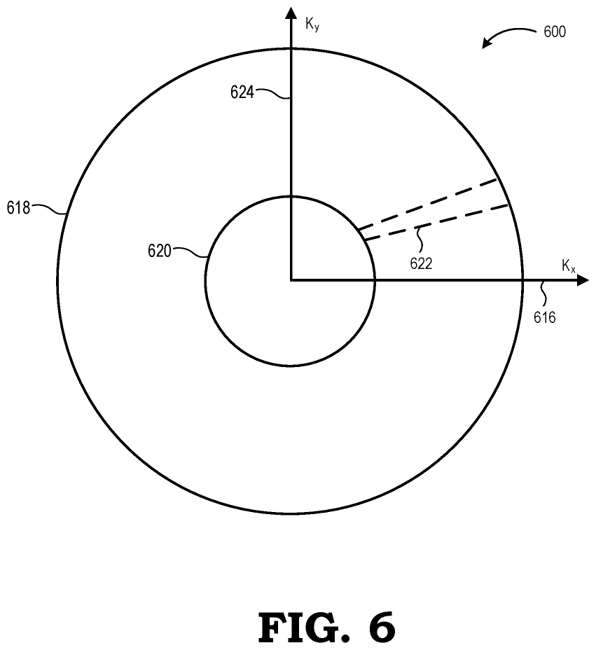

According to some embodiments, light sources 406 are disposed a distance (e.g., 50 mm) away from one or more test structures 404 such that the light 514 provided by the light sources 406 to one or more test structures 404 (e.g., via wafer 402) is collimated along one or more dimensions (e.g., axes). For example, a light source 406 is disposed a distance away from a test structure 404 such that the light 514 provided from the light source 406 to the test structure 404 via wafer 402 is collimated along a y axis. In this way, the light 514 received by a test structure 404 is limited to a single plane of incidence, reducing the complexity of the testing process for on-wafer test mechanism 400. Referring now to FIG. 6, an example k-space mode diagram 600 demonstrating an example collimation of light within on-wafer test mechanism 400 is presented, according to some embodiments. The k-space mode diagram 600 includes a first axis 624 representing spatial frequency information in a first dimension (e.g., K_{y}) and a second axis 616 representing spatial frequency information in a second dimension (e.g., Kₓ). According to an example embodiment, the k-space mode diagram 600 represents the degree of collimation of the modes of light 514 transmitted from a source 620 (e.g., a light source 406) and received at structure 618 (e.g., the test gratings 410 of a test structure 404). The modes of light 514, for example, represent the paths, bounces, angles, or any combination thereof beams of light 514 take as the beams of light 514 propagate through wafer 402 before being received at a test structure 404. Further, the degree of collimation of the modes of light 514 indicates a level of parallelism between the modes of light 514. To represent the degree of collimation of the modes of light 514, k-space mode diagram 600 includes slice 622 representing the degree of collimation of the modes of light 514. For example, the thickness of slice 622 represents the degree of collimation of the modes of light 514 with a greater thickness indicating, as an example, a lesser degree of collimation and a lesser thickness indicating, as an example, a greater degree of collimation.

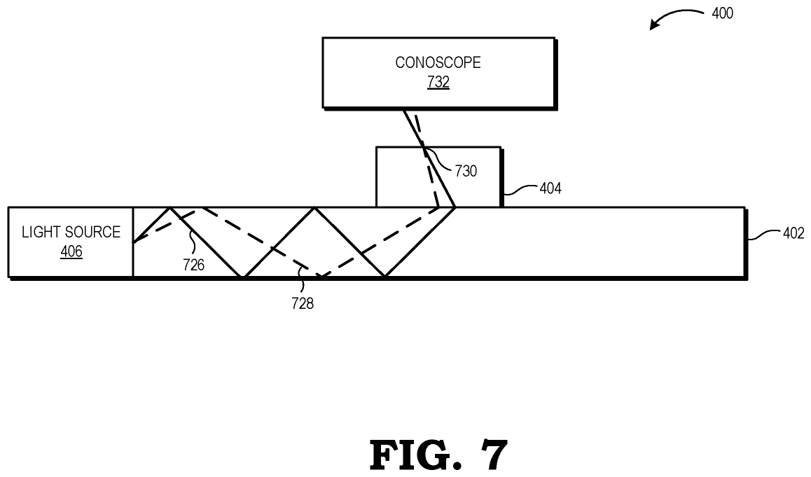

Referring now to FIG. 7, a cross-sectional view of an on-wafer test mechanism 400 is presented. In embodiments, one or more light sources 406 are configured to provide a beam of light 514 to a test structure 404 by propagating the light through wafer 402. That is to say, one or more light sources 406 emit a beam of light 514 such that the beam of light 514 propagates through wafer 402 and is received at a test structure 404. According to embodiments, as the beam of light 514 propagates through wafer 402, a first portion 726 of the beam of light 514 propagates through wafer 402 at a first mode (e.g., angle) and a second portion 728 of the beam of light 514 propagates through wafer 402 at a second mode (e.g., angle). For example, one or more light sources 406 emit light with the same wavelength (e.g., same color) that propagates through wafer 402 at a first mode and a second mode. In response to receiving the first portion 726, second portion 728, or both of light 514, a test structure 404 is configured to guide the first portion 726, second portion 728, or both to an exit point 730 on test structure 404 such that the first portion 726, second portion 728, or both are directed out of wafer 402. In embodiments, a portion 726, 728 exits the exit location 730 (e.g., exits wafer 402) at an angle based on the respective mode at which the portion 726, 728 propagated through wafer 402, the angle of one or more test gratings 410 of test structure 404, or both. That is to say, upon hitting the test grating 410 of a test structure 404, a portion 726, 728 of light diffracts at a distinct angle based on the respective mode at which the portion 726, 728 propagated through wafer 402 and the angle of the test grating 410.

In embodiments, as a portion 726, 728 exits exit location 730 (e.g., exits wafer 402), conoscope 732 is configured to take a measurement of the portion 426, 428 of light. Conoscope 732, for example, includes hardware-based circuitry, software-based circuitry, or both configured to measure the intensity, angle, or both of light. As an example, conoscope 732 is configured to measure the intensity of a portion 726, 728 as it exits wafer 402, the angle at which a portion 726, 728 exits wafer 402, or both. In embodiments, after measuring the intensity of a portion 726, 728 as it exits wafer 402 at the exit point 730 of a test structure 404, conoscope 732 is configured to determine the diffraction efficiency of the test gratings 410 of the test structure 404. For example, conoscope 732 is configured to determine the diffraction efficiency of the test gratings 410 based on a ratio of the measured intensity to the intensity at which one or more light sources 406 emit beams of light 514. After determining the diffraction efficiency of a set of test gratings 410, conoscope 732 is configured to determine the fidelity of the structures on wafer 402 (e.g., the test gratings 410 of the test structures 404 and the gratings of the waveguides 408 of the wafer 402), for example, by comparing the determined diffraction efficiency to a predetermined threshold value. As an example, in response to the determined diffraction efficiency being equal to or exceeding a predetermined threshold value, the conoscope 732 determines the structures on wafer 402 (e.g., the test gratings 410 of the test structures 404 and the gratings of the waveguides 408 of the wafer 402) have a high fidelity. Further, in response to the determined diffraction efficiency being less than the predetermined threshold value, conoscope 732 determines the structures on wafer 402 (e.g., the test gratings 410 of the test structures 404 and the gratings of the waveguides 408 of wafer 402) have a low fidelity.

According to some embodiments, conoscope 732 is configured to measure the light (e.g., portions 726, 728 of light 514) that exit a test structure 404 at a wide range of angles and map such angles to the modes (e.g., angles) at which the portions 726, 728 propagated through wafer 402 before exiting the wafer 402 at the exit point 730 of the test structure 404. In this way, conoscope 732 is configured to concurrently measure light exiting a test structure 404 at multiple angles, allowing conoscope 732 to measure the diffraction efficiency of the test gratings 410 of a test structure 404 at different angles concurrently. That is to say, conoscope 732 is configured to determine the diffraction efficiency of a set of test gratings 410 at multiple angles concurrently. Because conoscope 732 is configured to determine the diffraction efficiency of a set of test gratings 410 at multiple angles concurrently, the time and cost to test (e.g., determined the fidelity of) the structures on wafer 402 (e.g., the test gratings 410 of the test structures 404 and the gratings of the waveguides 408 of the wafer 402) is reduced.

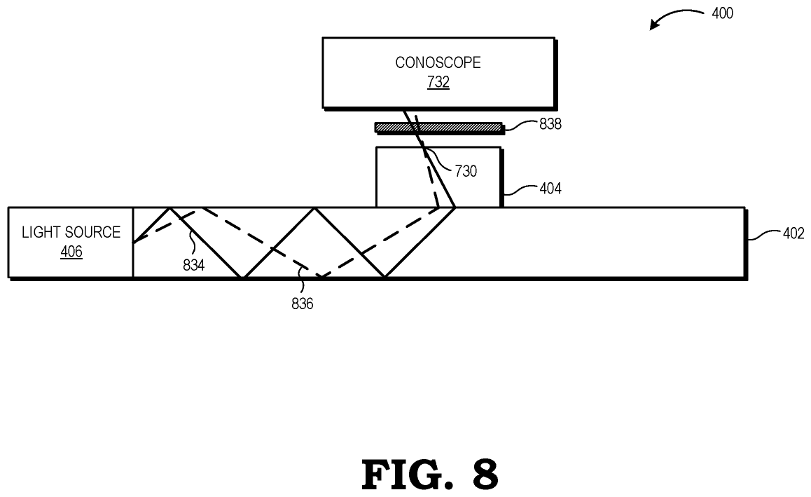

Referring now to FIG. 8, a cross-sectional view of an on-wafer test mechanism 400 is presented. In embodiments, one or more light sources 406 are configured to provide a first beam of light 834 (e.g., white light, red light, green light, blue light) having a first wavelength and a second beam of light 836 having a second, different wavelength to a test structure 404 by propagating the beams of light 834, 836 through wafer 402. That is to say, one or more light sources 406 emit a first beam of light 834 having a first wavelength and a second beam of light 836 having a second wavelength such that the beams of light 834, 836 propagate through wafer 402 and are received by a test structure 404. In response to receiving a beam of light 834, 836, the test gratings 410 of a test structure 404 are configured to guide the received beam of light 834, 836 to an exit location 730 of the test structure 404 and out of the wafer 402. As a beam of light 834, 836 exits wafer 402, conoscope 732 measures the beam of light 834, 836. As an example, conoscope 732 is configured to measure the intensity of the beam of light 834, 836 as it exits wafer 402. In embodiments, after measuring the intensity of the beam of light 834, 836 as it exits wafer 402, conoscope 732 is configured to determine the diffraction efficiency of the test gratings 410 of the test structure 404 based on the measured intensity (e.g., based on a ration of the measured intensity to the intensity of the beam of light 834, 836 when emitted by a light source 406).

After determining the diffraction efficiency of a set of test gratings 410, conoscope 732 is configured to determine the fidelity of the structures on wafer 402 (e.g., the test gratings 410 of the test structures 404 and the gratings of the waveguides 408 of the wafer 402) by comparing the determined diffraction efficiency to a predetermined threshold value. According to some embodiments, conoscope 732 includes one or more filters (e.g., narrow bandpass filters) configured to allow conoscope 732 to differentiate between wavelengths of light by filtering one or more wavelengths of light. In this way, conoscope 732 is configured to concurrently determine the diffraction efficiency of a set of test gratings 410 at multiple wavelengths. Because conoscope 732 is configured to determine the diffraction efficiency of a set of test gratings 410 at multiple wavelengths concurrently, the time and cost to test (e.g., determined the fidelity of) the structures on wafer 402 (e.g., the test gratings 410 of the test structures 404 and the gratings of the waveguides 408 of the wafer 402) is reduced.

In some embodiments, on-wafer test mechanism 400 further includes a linear polarizer 838 disposed between test structure 404 on wafer 402 and conoscope 732 configured to polarize light in a first linear direction (e.g., vertical direction), a second linear direction (e.g., horizontal direction), or both perpendicular to the axis of a beam of light. Such a linear polarizer 838 includes, for example, a polarizing film (e.g., a polyvinyl alcohol polarizing film) configured to polarize received light in a linear direction (e.g. first linear direction, second linear direction) perpendicular to the axis of a beam of light. Using linear polarizer 838, conoscope 732 is configured to separately measure the diffraction efficiency of a set of test gratings 410 at different polarization states. For example, conoscope 732 is configured to measure the diffraction efficiency of a set of test gratings 410 at two distinct polarization states, as an example, x-polarized and y-polarized. As an example, when a test structure 404 is disposed a distance away from a light source 406 such that the light provided by light source 406 to the test structure 404 is collimated along one or more dimensions and test structure 404 is disposed at a same position along a dimension (e.g., y-dimension) as the light source 406, no change in the polarization of the light provided by light source 406 to test structure 404 occurs. As such, when such light provided by light source 406 exits wafer 402, linear polarizer 838 polarizes the light in a first linear direction such that conoscope 732 measures the intensity of the light having a first polarization state. Further, in some embodiments, linear polarizer 838 polarizes the light in a second linear direction such that conoscope 732 measures the intensity of the light having a second polarization state. In this way, conoscope 732 is configured to measure the diffraction efficiency of a set of test gratings 410 at specific input polarization states.

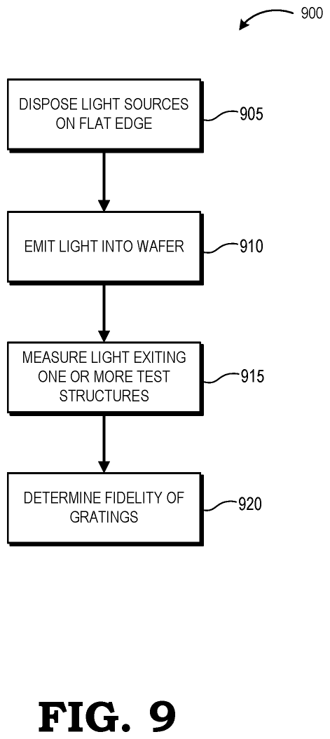

Referring now to FIG. 9, an example method 900 for testing the fidelity of the gratings (e.g., structures) on a wafer is presented, in accordance with some embodiments. For example, step 905 of example method 900 includes one or more light sources 406 being disposed proximate to or otherwise coupled to the flat edge 412 of a wafer 402 such that light emitted from the light sources 406 propagates through wafer 402. At step 910, the light sources 406 emit light into wafer 402 and the light propagates through wafer 402 at one or more modes (e.g., angles). At step 915, at least a portion of the light propagating through wafer 402 is received at a test structure 404 of wafer 402. For example, at least a portion of the light is received by a set of test gratings 410 of the test structure 404. In response to receiving the portion of light, the test gratings 410 guide the received light to an exit point 730 of the test structure 404 such that the received light is directed out of wafer 402. Further, at step 915, conoscope 732 is configured to take one or more measurements of the light exiting wafer 402. For example, conoscope 732 measures the angles, intensities, or both of the light exiting wafer 402.

At step 920, conoscope 732 determines the fidelity of the gratings (e.g., test gratings 410 of the test structures and the gratings of the waveguide 408) on wafer 402. To this end, conoscope 732 determines the intensity of the light exiting wafer 402 at one or more angles (e.g., modes). For example, conoscope 732 measures the intensity of the light exiting wafer 402 at one or more measured angles and then determines a mode for each measured angle such that conoscope 732 determines an intensity for each determined mode of the light. Further conoscope 732 is configured to determine a diffraction efficiency for the test gratings 410 that directed the light out of wafer 402. As an example, conoscope 732 determines a diffraction efficiency based on a ratio of the measured intensity to the intensity at which the light sources 406 emitted the light. In embodiments, conoscope 732 is configured to determine a respective diffraction efficiency for a set of test gratings 410 for each determined mode of the light exiting wafer 402. To determine the fidelity of the gratings (e.g., test gratings 410 of the test structures and the gratings of the waveguide 408) on wafer 402, conoscope 732 compares one or more of the determined diffraction efficiencies to one or more predetermined threshold values. In response to one or more of the determined diffraction efficiencies being equal to or exceeding one or more predetermined threshold values, the conoscope 732 determines that the gratings (e.g., test gratings 410 of the test structures and the gratings of the waveguide 408) on wafer 402 have a less than one or more predetermined threshold values, the conoscope 732 determines that the gratings (e.g., test gratings 410 of the test structures and the gratings of the waveguide 408) on wafer 402 have a low fidelity.

In some embodiments, certain aspects of the techniques described above may be implemented by one or more processors of a processing system executing software. The software comprises one or more sets of executable instructions stored or otherwise tangibly embodied on a non-transitory computer-readable storage medium. The software can include the instructions and certain data that, when executed by the one or more processors, manipulate the one or more processors to perform one or more aspects of the techniques described above. The non-transitory computer-readable storage medium can include, for example, a magnetic or optical disk storage device, solid-state storage devices such as Flash memory, a cache, random access memory (RAM) or other non-volatile memory device or devices, and the like. The executable instructions stored on the non-transitory computer-readable storage medium may be in source code, assembly language code, object code, or other instruction format that is interpreted or otherwise executable by one or more processors.

A computer-readable storage medium may include any storage medium, or combination of storage media, accessible by a computer system during use to provide instructions and/or data to the computer system. Such storage media can include, but is not limited to, optical media (e.g., compact disc (CD), digital versatile disc (DVD), Blu-ray disc), magnetic media (e.g., floppy disc, magnetic tape, or magnetic hard drive), volatile memory (e.g., random access memory (RAM) or cache), non-volatile memory (e.g., read-only memory (ROM) or Flash memory), or microelectromechanical systems (MEMS)-based storage media. The computer-readable storage medium may be embedded in the computing system (e.g., system RAM or ROM), fixedly attached to the computing system (e.g., a magnetic hard drive), removably attached to the computing system (e.g., an optical disc or Universal Serial Bus (USB)-based Flash memory), or coupled to the computer system via a wired or wireless network (e.g., network accessible storage (NAS)).

Note that not all of the activities or elements described above in the general description are required, that a portion of a specific activity or device may not be required, and that one or more further activities may be performed, or elements included, in addition to those described. Still, further, the order in which activities are listed is not necessarily the order in which they are performed. Also, the concepts have been described with reference to specific embodiments. However, one of ordinary skill in the art appreciates that various modifications and changes can be made without departing from the scope of the present disclosure as set forth in the claims below. Accordingly, the specification and figures are to be regarded in an illustrative rather than a restrictive sense, and all such modifications are intended to be included within the scope of the present disclosure.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims. Moreover, the particular embodiments disclosed above are illustrative only, as the disclosed subject matter may be modified and practiced in different but equivalent manners apparent to those skilled in the art having the benefit of the teachings herein. No limitations are intended to the details of construction or design herein shown, other than as described in the claims below. It is therefore evident that the particular embodiments disclosed above may be altered or modified and all such variations are considered within the scope of the disclosed subject matter. Accordingly, the protection sought herein is as set forth in the claims below.

What is claimed is:

1. A wafer, comprising:
a test structure disposed on the wafer; and
a light source configured to provide a beam of light to the test structure, wherein:
the beam of light is configured to propagate through the wafer, and
the test structure is configured to guide at least a portion of the beam of light out of the wafer.

2. The wafer of claim 1, wherein the test structure includes a set of gratings configured to direct the at least a portion of the beam of light out of the wafer.

3. The wafer of claim 1, further comprising:
a conoscope configured to measure an intensity of the at least a portion of the beam of light as the at least a portion of the beam of light exits the wafer.

4. The wafer of claim 3, wherein the conoscope is further configured to:
determine a diffraction efficiency of the test structure based on the measured intensity.

5. The wafer of claim 1, further comprising:
a second light source configured to provide a second beam of light to the test structure, wherein the second beam of light is configured to propagate through the wafer.

6. The wafer of claim 5, wherein the beam of light includes a first wavelength and the second beam of light includes a second wavelength that is different from the first wavelength.

7. The wafer of claim 6, further comprising:
a conoscope configured to determine a first diffraction efficiency of the test structure at the first wavelength and a second diffraction efficiency at the second wavelength.

8. The wafer of claim 1, wherein the light source is coupled to a flat edge of the wafer.

9. The wafer of claim 1, wherein the light source is disposed proximate to a flat edge of the wafer.

10. A wafer, comprising:
a test structure disposed on the wafer; and
a plurality of light sources configured to provide light to the test structure,
wherein:
the light is configured to propagate through the wafer, and
the test structure is configured to guide at least a portion of the light out of the wafer.

11. The wafer of claim 10, further comprising:
a conoscope configured to:
determine one or more modes of the light propagating through the wafer; and
determine a respective diffraction efficiency of the test structure for each mode of the one or more modes of light propagating through the wafer.

12. The wafer of claim 11, further comprising:
a linear polarizer disposed between the test structure and the conoscope.

13. The wafer of claim 10, wherein the test structure includes a set of gratings configured to direct the at least a portion of the light out of the wafer.

14. The wafer of claim 10, wherein the plurality of light sources is coupled to a flat edge of the wafer.

15. The wafer of claim 10, wherein the plurality of light sources is disposed proximate to a flat edge of the wafer.

16. The wafer of claim 10, wherein the plurality of light sources are configured to activate sequentially.

17. A method, comprising:
providing, from a light source, light to a test structure disposed on a wafer, wherein the light is configured to propagate through at least a portion of the wafer; and
measuring an intensity of at least a portion of the light exiting the wafer at the test structure; and
determining a diffraction efficiency of the test structure based on the measured intensity.

18. The method of claim 17, further comprising:
determining one or more modes of the light propagating through the wafer; and
determining a respective diffraction efficiency of the test structure for each of the one or more modes.

19. The method of claim 17, wherein the light source is coupled to a flat edge of the wafer.

20. The method of claim 17, wherein the light source is disposed proximate to a flat edge of the wafer.
